(19) United States
(12) Patent Application Publication
(10) Pub. No.: US 2014/0113538 A1
(43) Pub. Date: Apr. 24, 2014

(54) DUAL AXIS VENT ROD AIR FRESHENER

(71) Applicant: AMERICAN COVERS, INC., Draper, UT (US)

(72) Inventors: Aaron Irvin, Salt Lake City, UT (US); Nate Finlay, Lehi, UT (US)

(73) Assignee: American Covers, Inc., Draper, UT (US)

(21) Appl. No.: 13/798,912

(22) Filed: Mar. 13, 2013

Related U.S. Application Data
(60) Provisional application No. 61/717,506, filed on Oct. 23, 2012.

(51) Int. Cl. B60H 3/00; A61L 9/12

(52) U.S. Cl. CPC B60H 3/0028; A61L 9/12
USPC 454/156; 239/60

(57) ABSTRACT

An air freshener includes a dual axis vent stick with a pair of transverse slots bifurcating the vent stick to form four fingers oriented substantially parallel to one another, and also bifurcating at least one scented body carried by the vent stick.

### PRIORITY CLAIM(S)

Priority is claimed to U.S. Provisional Patent No. 61/717,506, filed Oct. 23, 2012, which is hereby incorporated herein by reference.

### RELATED APPLICATIONS

This is related to U.S. Design patent application Ser. No. 29/435,389, filed Oct. 23, 2012, which is hereby incorporated herein by reference.

This is related to U.S. Pat. Nos. 7,687,038; 7,687,037; 8,147,761; D594,954; D640,359; D650,892; and U.S. patent application Ser. Nos. 13/009,574, filed Jan. 19, 2011; 12/915,983, filed Oct. 29, 2010; 13/359,726, filed Jan. 27, 2012; and U.S. Provisional Patent Application Ser. No. 61/566,161; which are all hereby incorporated herein by reference.

### BACKGROUND

1. Field of the Invention

The present invention relates generally to air fresheners.

2. Related Art

Air fresheners are common devices used to improve and/or change the olfactory characteristics of an environment. Such environments can include bathrooms or wash closets, vehicles, lockers, drawers, etc. Such air fresheners typically include a scent that is aesthetically pleasing, such as flowers, fruits, etc.

One common type of air freshener is a two-dimensional, paper-fiber card with a fragrance surrounded in a clear plastic envelope. The envelope is pierced to form an opening, and a portion of the freshener protrudes through the opening to release a scent. The air freshener includes a string or elastic forming a loop to suspend the air freshener. Such air fresheners are commonly utilized in vehicles. One disadvantage with such air fresheners is that the card is moist, and can leak or leach, leaving a stain on other surfaces, such as dashboards. The plastic envelope and the suspending loop act to resist contact between the card, and other surfaces. Another disadvantage with such air fresheners is that they are aesthetically displeasing. Another disadvantage with such air fresheners is that they have a more pronounced and immediate scent release, releasing a majority of the scent in the first few days of use.

Another common type of air freshener has a disc-shaped shell or canister with a scented disc or gel inside. The shell can include an adhesive strip to stick or adhere the shell to a surface. One disadvantage with such air fresheners is that removal of the shell often leaves a residue of the adhesive on the surface, which is aesthetically displeasing. In addition, the scented material can leak and harm the surface.

Some disadvantages of common air fresheners include 1) rapid scent loss or lack of longevity; 2) non-linear or inconsistent scent release over time; and 3) risk of staining. Some air fresheners disperse their scent too rapidly, thus losing effectiveness over time, and not lasting as long as desired. Some air fresheners quickly or rapidly disperse their scent after activation, and then slowly release scent, or release little scent, thereafter. Some air fresheners include materials that can leak and stain.

Another disadvantage with some air fresheners is that they include a non-solid and non-liquid material, or a jelly-like material, that must be contained in some type of container because the jelly is flowable. The material can become dried and cracked over time, presenting an aesthetically displeasing appearance. Again, the scented material can leak and damage surfaces.

### SUMMARY OF THE INVENTION

It has been recognized that it would be advantageous to develop an air freshener that is more aesthetically pleasing. In addition, it has been recognized that it would be advantageous to develop an air freshener that is easily located or positioned without marring or otherwise damaging other surfaces.

The invention provides an air freshener air freshener with an elongated vent stick configured to extend into an air vent of a vehicle and having a proximal end, a distal end, and a longitudinal axis. At least one scented body is carried by the vent stick and is configured to extend into the air vent. A scent material is interspersed throughout the at least one scented body and is capable of dispersing out of the at least one body over time. A pair of transverse slots is oriented transverse to one another, and each bifurcates the vent stick in a different direction to form four fingers oriented substantially parallel to one another.

In accordance with a more detailed aspect of the present invention, each of the pair of slots can have a different length with respect to one another from the distal end of the vent stick. The pair of slots can include: a primary slot bifurcating only the vent stick to form a pair of vent rods forming a clip; and a secondary slot bifurcating both the pair of vent rods of the vent stick and the at least one body. The at least one scented body can further include a pair of scented bodies, each carried by a different one of the pair of vent rods, and with the primary slot separating the pair of bodies and the secondary slot bifurcating the pair of scented bodies. The air freshener can further include a head formed on the proximal end of the vent stick and having a face configured to face outward from the air vent. The face can have a concave cascading curvature from a top thereof to a bottom thereof. The face can have a lateral concave indentation oriented concave from side to side across the face. The cascading curvature can be convex from the top of the face to the bottom of the face. A channel can be formed in the bottom of the head behind the face and oriented transverse to the longitudinal axis of the vent stick. One of the pair of transverse slots can be oriented horizontally while another of the pair of transverse slots can be oriented vertically.

In addition, the invention provides an air freshener in combination with an air vent of a vehicle. The air freshener includes a vent stick extending into and engaging the air vent and having a proximal end, a distal end, and a longitudinal axis. A pair of scented bodies is carried by the vent stick and extending into the air vent. A scent material is interspersed within and throughout the pair of scented bodies and capable of dispersing out of the pair of scented bodies over time. A primary slot oriented horizontally and extends into the vent stick from the distal end and bifurcates the vent stick to form a pair of vent rods forming a clip and separating the pair of scented bodies with each carried by a different one of the pair of vent rods. A secondary slot is oriented vertically and extends into the vent stick from the distal end and is oriented transverse to the primary slot and bifurcates the pair of vent rods and the pair of scented bodies to form four fingers oriented substantially parallel to one another.

Furthermore, the invention provides an air freshener including an elongated vent stick configured to extend into an air vent of a vehicle and having a proximal end, a distal end, and a longitudinal axis. A scent material is associated with the vent. A pair of transverse slots is oriented transverse to one another and each bifurcates the vent stick in different direction to form four fingers oriented substantially parallel to one another. A head is formed on the proximal end of the vent stick and has a face configured to face outward from the air vent.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1 is a perspective view of an air freshener with a dual axis vent stick with a pair of transverse slots bifurcating the vent stick to form four fingers oriented substantially parallel to one another, and also bifurcating at least one scented body carried by the vent stick, in accordance with one aspect of the present invention;

FIG. 2 is a side view of the air freshener of FIG. 1;

FIG. 3 is a top view of the air freshener of FIG. 1;

FIG. 4 is a bottom view of the air freshener of FIG. 1;

FIG. 5 is a front end view of the air freshener of FIG. 1;

FIG. 6 is a rear end view of the air freshener of FIG. 1;

FIG. 7 is a perspective view of another air freshener in accordance with another embodiment of the present invention;

FIG. 8 is a side view of the air freshener of FIG. 7;

FIG. 9 is a top view of the air freshener of FIG. 7;

FIG. 10 is a bottom view of the air freshener of FIG. 7;

FIG. 11 is a rear perspective view of the air freshener of FIG. 7;

FIG. 12 is a cross-sectional side perspective view of the air freshener of FIG. 7 taken along line 12 of FIG. 10;

FIG. 13 is a cross-sectional side perspective view of the air freshener of FIG. 7 taken along line 13 of FIG. 10;

FIG. 14 is a cross-sectional side view of the air freshener of FIG. 7 taken along line 12 of FIG. 10; and

FIG. 15 is a cross-sectional end perspective view of the air freshener of FIG. 7 taken along line 15 of FIG. 10

Reference will now be made to the exemplary embodiments illustrated, and specific language will be used herein to describe the same. It will nevertheless be understood that no limitation of the scope of the invention is thereby intended.

### DETAILED DESCRIPTION OF EXAMPLE EMBODIMENT(S)

### Definitions

The term “scent material” and “fragrant material” are used interchangeably herein to refer broadly to a material that carries a desired fragrance or scent that can be discernable or smelled, or even a neutralizing agent. Thus, the scent or fragrance can be an ascertainable smell used to cover other scents, or a neutral agent that eliminates odors or provides a fresher atmosphere.

The terms “horizontal” and “vertical” are used herein relative to one another.

### Description

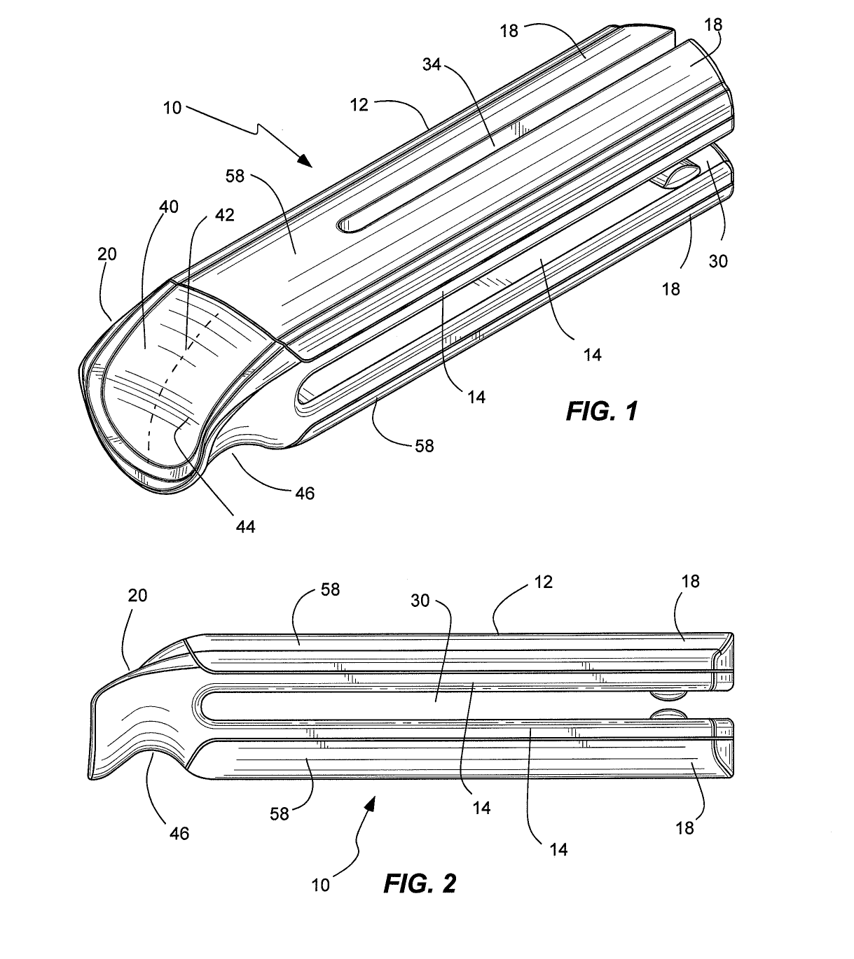

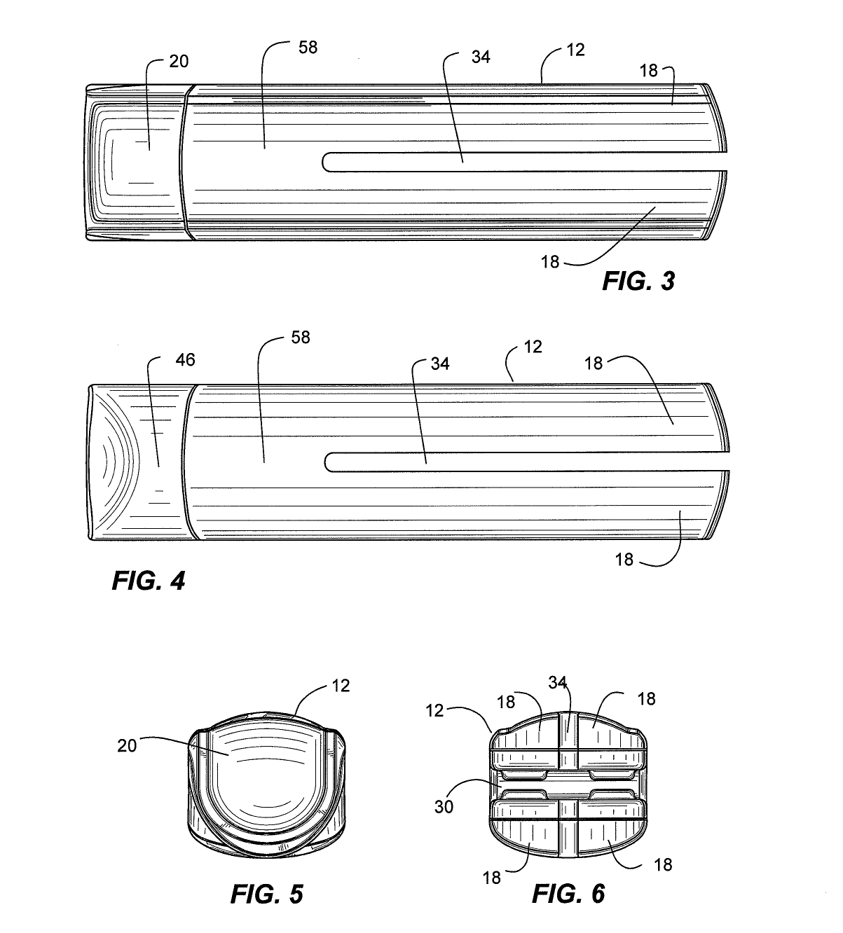

As illustrated in FIGS. 1-6, an air freshener or scent device, indicated generally at 10, in accordance with the present invention is shown for providing a desired and/or aesthetically pleasing scent, fragrance, aroma or neutralizing agent. Air fresheners are one example of a field that can benefit from the present invention. The air freshener 10 can be configured for use in air vents of vehicles, and can also be used in bathrooms, wash closets, offices, bedrooms, etc, and with air vents in other locations or of different types. The desired scent can include, for example, floral, fruit, vanilla, berry, pine, etc. The air freshener can have a dual axis stick design with a pair of perpendicular slots (horizontal and vertical) and creating four fingers, to facilitate engaging the louvers of the air vent. An air vent can have primary or outer louvers oriented substantially horizontally, and can also have secondary or inner louvers behind the primary or outer louvers that are oriented vertically. In addition, an air vend can have both primary and secondary louvers, oriented horizontally and vertically, respectfully, that intersect one another to form a grid.

The air freshener 10 can include an elongated vent stick 12 to extend into an air vent and having a proximal end, a distal end, and a longitudinal axis. The vent stick can include one or more rods or vent rods 14 forming a clip to engage the air vent, or grid or louvers thereof. The one or more vent rods 14 can include one or more fingers 18 that can also form a clip to engage the air vent, or grid or louvers thereof. The vent stick 12 can have a length configured to extend into the air vent or duct behind the air vent. In addition, the vent stick 12 can have a width and a height sized to pass through the air vent or louvers. The width and the height can be sized less than the length. The vent stick 12 can have the proximal end towards the user and at the gird or louvers of the air vent, the distal end in the air vent, and can have or can define the longitudinal axis oriented along the length or long dimension thereof so that the longitudinal axis extends into and from the air vent. The vent stick 12 can be formed of plastic, and can be substantially or relatively rigid, but also flexible and resilient so that the distal end thereof can expand about the grid or louvers.

A scent material with a desired scent or fragrance can be associated with and carried by the vent stick. The scent material can be carried by the vent stick, or vent rods or fingers thereof. In addition, the scent material can be interspersed without and throughout a scented body(ies) 58, such as a polymer body or carrier material carried by the vent stick. The body(ies) 58 or the carrier material or polymer body can have a size and shape similar to the size and shape of the vent stick, vent rods and fingers. Thus, the body(ies) 58 or the polymer body can extend along an exterior of the stick, rods and/or fingers for substantially the length thereof. The scented body 58 can include multiple bodies, each attached to a different rod or finger. In another aspect, the scented body can be a single body that extends along one rod or finger, over a proximal end (or head) of the stick, and back along another rod or finger. The scented body or polymer body can have an exposed side or surface with indicia thereon. The scented body or carrier material or polymer body can be coupled to the stick/rods/fingers and extend into the air vent therewith. In one aspect, the stick or rods or fingers can have a plurality of protrusions 62 spaced at predetermined intervals along the length thereof, and the scented body or carrier material or polymer body can have a plurality of apertures 66 therein corresponding to the protrusions. In another aspect, the protrusions can be formed in the body, and the apertures can be formed in the rods. The scented body or carrier material or polymer body can be coupled to the stick, rods or fingers by snapping the apertures onto the protrusions. In this way, the scented body or carrier material or polymer body can be inserted with the stick, rods or fingers into the air vent to increase scent dispersal as air blows through the vent and through or around the scented body. The stick and scented body together can define a body of the air freshener with respect to a head thereof.

The scented body or polymer body can be flexible and resilient, such as a polymer gel. The scented or polymer body can be elastic and coherent. Thus, the scented or polymer body can be compressible under an applied force, and substantially returnable to an original configuration upon removal of the applied force. It has been found that the polymer gel provides desired characteristics of aesthetics, flexibility, longevity, substantially constant scent release, and containment. In accordance with another aspect of the present invention, the polymer gel can have a freestanding, self-supported, three-dimensional shape that does not significantly change as the scent is released. The scented body or carrier material or polymer body can have a scent material of the desired scent interspersed therein. The scent material disperses or diffuses out of the scented body or carrier material or polymer body into the air or atmosphere where it can be detected, or where it can provide a discernable scent. It is believed that the scent material migrates or diffuses through and out of the carrier material or polymer body. The scent material can be high in volatile notes, or has high volatility and can vaporize or evaporate at low temperatures. The scent material can include a scented oil. For example, suitable scent material can include pine, berry, vanilla, apple, coconut, cherry, pina colada, etc.

The scented body or carrier material or polymer body can include a polymer material, such as a polymer gel. The polymer body and/or polymer gel can be elastic and coherent. Thus, the scented or polymer body can elastically deform under normal conditions. The scented or polymer body can be flexible and resilient, such that the body or gel can compress under an applied force, but can substantially return to its original configuration upon removal of the applied force. The polymer gel, or the polymer body, can have a freestanding, self-supported, three-dimensional shape. Thus, the polymer gel or polymer body can be consistent or solid enough to support or maintain its shape in a freestanding manner without a container. The three-dimensional shape can be any desired shape. The polymer gel can be considered a solid material that is elastic and coherent, and thus flexible and capable of being deformed, but without being flowable. Thus, the polymer gel may have a sufficiently high molecular weight, and/or a sufficiently high viscosity, so that it is a non-flowable gel. In addition, the polymer gel can be considered as stable. Thus, the polymer gel can be bendable, but otherwise substantially maintains its form. The scented body or polymer body or polymer gel can be characterized as a polymeric material in the glass state with substantially no macroscopic flow. The polymeric material can have a glass transition temperature greater than approximately 110° F. The polymer gel can retain its gel-like characteristic over time, without drying or cracking, and without becoming hard or brittle.

In one embodiment, the polymer gel or scented or polymer body can include a polyurethane material or can be a polyurethane gel. The gel can be formed by combining a polyurethane material with a scented oil. Surprisingly, it has been found that such a combination provides a desired scent, but without staining or substantially leaking onto a surface. In addition, the scented or polymer body does not undergo a visually ascertainable physical change, such as drying out or cracking. Thus, the scented or polymer body remains aesthetically pleasing.

In some embodiments, a scented oil and a polymerizable monomer can be combined, along with optional initiators or other reactants. Isocyanate reaction polymers have shown good results in connection with the present invention. For example, the polymer gel and scent material can be a urethane polymerization product of combining a scented oil with a polyether polyol, and then with a diphenylmethane diisocyanate (MDI) prepolymer. Therefore, in some embodiments, the scent material can be a scented oil which participates in the polymerization reaction between polymerization reactants. For example, essential oils such as terpenes and the like can be mixed with polymerization reactants, or even in place of some reactants. Without being bound by any particular theory, it is thought that at least some of the reduction or elimination in residue in the devices of the present invention result from at least partial replacement of mineral oils and/or polyols with scented oils such as those listed herein. Other suitable isocyanates can include, but are not limited to, tolylene diisocyanates, methylene diphenyl isocyanates, hexamethylene diisocyanates, prepolymers thereof, and the like. Those skilled in the art will recognize various other isocyanate reaction polymers, i.e. polyurethanes, which can be suitable for use in connection with the present invention.

Alternatively, the polymer gel or polymer body can include silicone, diffused polyurethane, polyvinylchloride (PVC), ethylene vinyl acetate (EVA), thermoplastic polyurethane (TPU), a polymer encapsulation fragrance delivery platform (PolyIFF®), thermoplastic elastomer (TPE), polypropylene, ethylene/methacrylic acid (E/MAA) copolymer, in which the MAA groups have been partially neutralized with lithium ions (Surlyn® Dupont), etc.

As a general guideline, the polymer gel or polymer body can be formed of an elastomer such as, but not limited to, urethanes (including polyester and polyether polyol/isocyanate polymerization products), polyacrylates, polybutadienes, ethylene propylene elastomers, silicones, natural and synthetic rubbers, styrene/butadiene block copolymers, and the like. In some embodiments, the polymer gel can be formed of a thermoplastic elastomer. Thermoplastic elastomers can be block copolymers such as polyurethanes, polyamides, copolyesters, and styrene-butadiene-styrene polymers. Other thermoplastic elastomers can be elastomer/thermoplastic blends such as ethylene-propylene-diene monomer in an isotactic polypropylene phase or nitrile rubber dispersed in a PVC phase. As used herein, “thermoplastic elastomer” refers to an elastomer which can be heated and processed like thermoplastic materials. Specifically, a thermoplastic elastomer can be heated to a melted or flowable state and then cooled, resulting in reformation of cross-linking and subsequent coherency without a substantial change in mechanical properties such as strength, flexural modulus, elastic modulus, etc.

As used herein, “elastomeric polymer” and “elastomer” may be used interchangeably and refer to a polymeric material which can be mechanically deformed and upon release returns to an original shape. A coherent elastomer is also non-flowable at or near room temperatures.

Although the polymer gel or scented or polymer body is freestanding and self-supporting, it is also flexible and resilient. Thus, scented body or polymer body or carrier material can form a flexible and resilient structure or body that can be selectively deformed and can return substantially to the three-dimensional shape. In addition, the scented body or carrier material or polymer body can be opaque. Alternatively, the scented body or carrier material or polymer body can be light transmissive in at least a translucent manner.

A pair of transverse slots 30 and 34 can be formed in the stick 12 to form the pair of rods 14 and the pair of fingers 18. In addition, at least one of the pair of slots 34 can be formed in the one or more bodies 58 carried by the pair of rods 14. The slots can be oriented transverse to one another and each can bifurcate the vent stick 12 to form four fingers 18 oriented substantially parallel to one another. The slots can have different lengths from the distal end of the vent stick. A primary slot 30 can be oriented horizontally and can bifurcate only the vent stick 12 to form the pair of vent rods 14 which form a clip. In addition, the primary slot can separate a pair of bodies 58. A secondary slot 34 can be oriented vertically and can bifurcate both of the pair of vent rods 14 of the vent stick 18, and the one or more bodies 58. Thus, the bodies 58 can be bifurcated as well. The pair of slots can be orthogonal or perpendicular to one another. The pair of slots can create a horizontal slot 30 and a vertical slot 34 to accommodate vent louver, grid or cover designs. In addition, bifurcating the bodies can create a greater surface area for increased scent release.

The air freshener 10 can also include a head 20 on a proximal end of the vent stick 12, and the rods 14 and/or fingers 18 can extend therefrom, or from a rear thereof. The head 20 and rods/fingers can be integrally formed together as a single monolithic body. The head 20 can have a face 40 configured to face outward from the air vent. The face 40 or head 20 can include indicia visible to the user. The face 40 can have a concave cascading curvature 42 from a top thereof to a bottom thereof. The face 40 can also have a lateral concave indentation 44 oriented concave from side to side across the face 40. The cascading curvature 42 can be convex from the top of the face to the bottom of the face. Thus, the face 40 can form a thumb grip or receptacle. Furthermore, the head 20 can have a channel 46 formed in the bottom of the head behind the face, and oriented transverse to the longitudinal axis of the vent stick. The channel can form a finger grip or receptacle. Thus, together, the thumb and finger grips or receptacles can provide a head that facilitates grasping for insertion and removal of the vent stick or air freshener from the louvers or cover of the air vent.

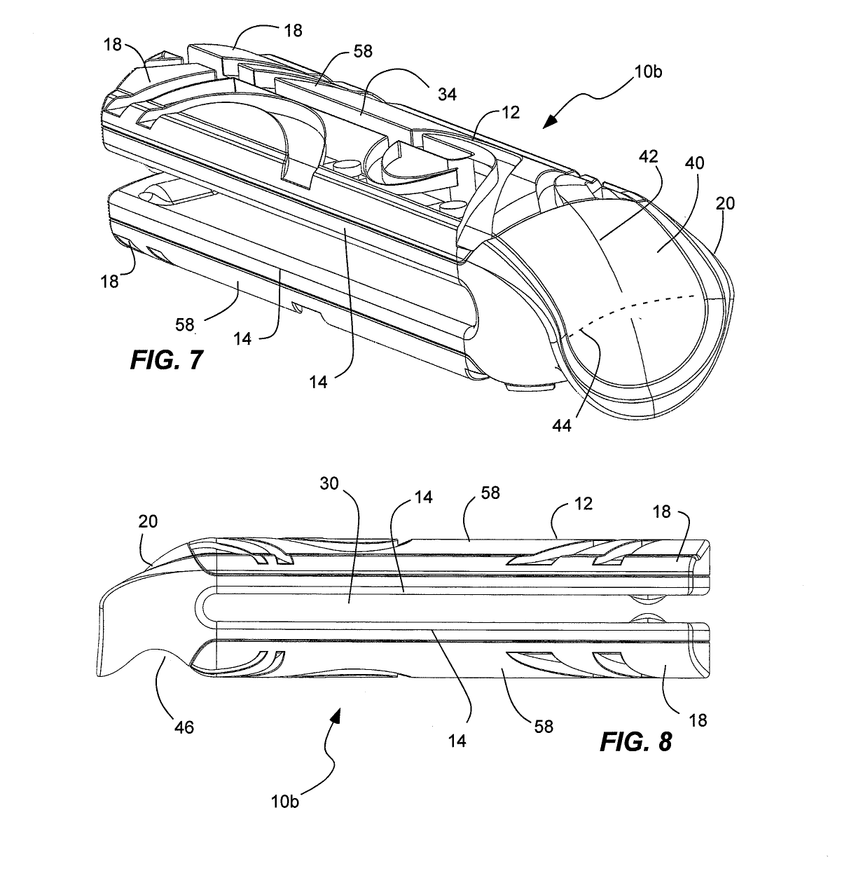

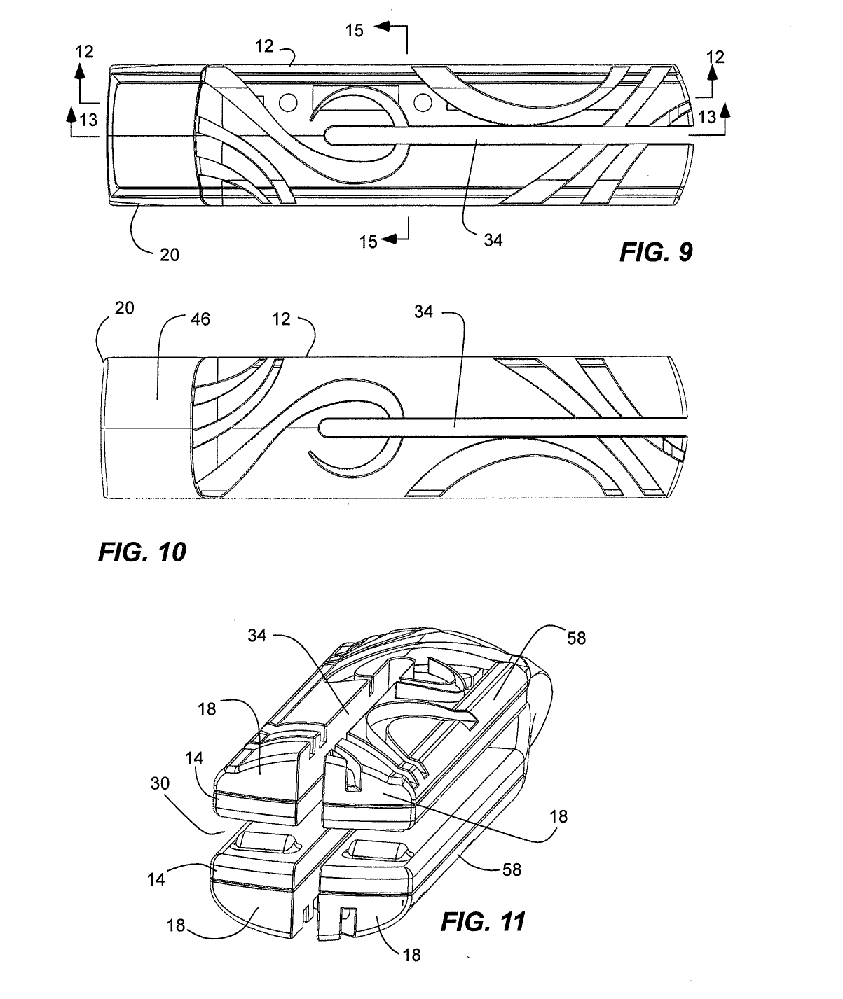

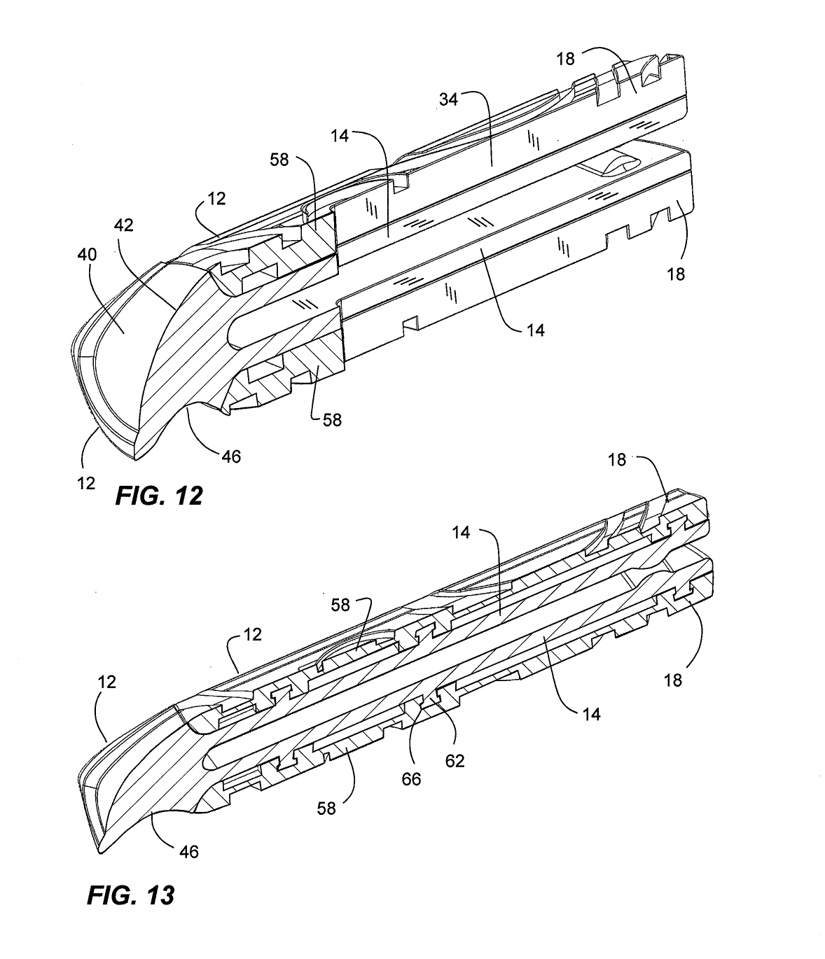

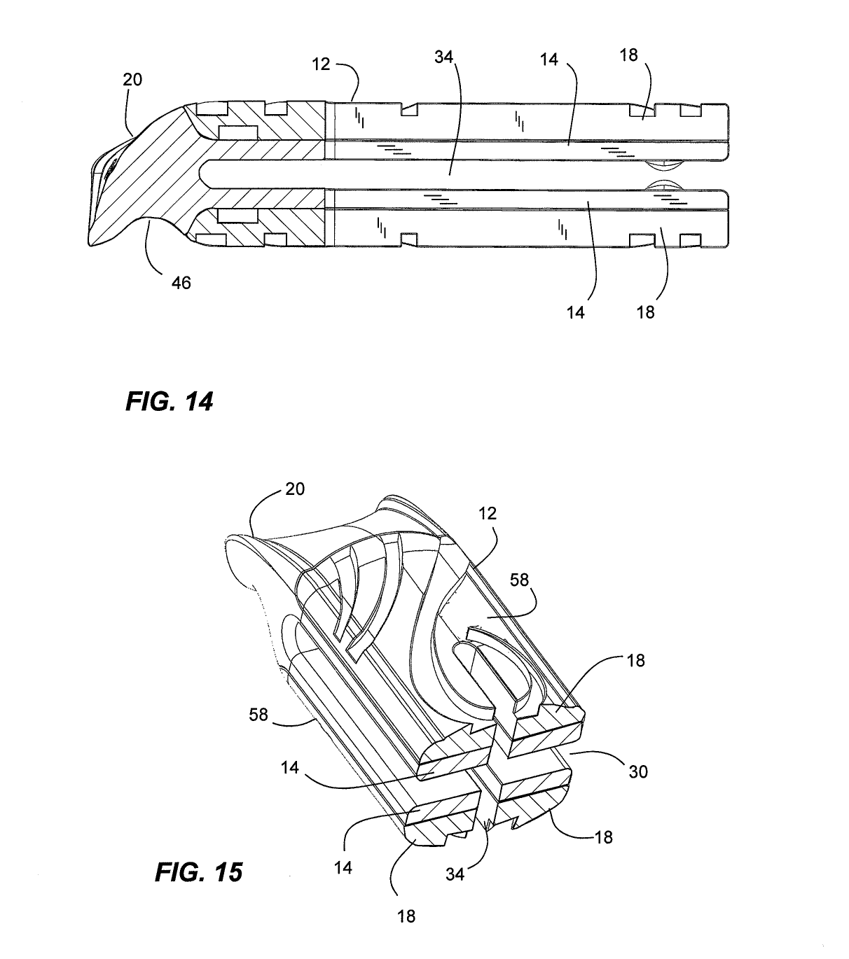

Referring to FIGS. 7-15, another air freshener 10b is shown that is similar in most respects to that described above, and which description is hereby incorporated herein by reference. The air freshener or scented bodies 58b can include indentations and/or protrusions formed therein to increase the surface area through which the scent can diffuse, and/or reduce the thickness of the body through which the scent must diffuse,.

In another aspect, the air freshener can also include a head pivotally coupled to the vent stick or rods. A hinge can be formed or defined between the head and the vent stick to allow the head to pivot with respect to the vent stick. The head and the vent stick can have multiple configurations with respect to one another, such as: a storage or packaged configuration with a face of the head substantially facing a direction perpendicular to the longitudinal axis of the vent stick, and an in use configuration with the face of the head facing in a direction substantially parallel to the longitudinal axis of the vent stick.

The hinge can include a clip or axel engaged in an aperture. For example, the clip or axel can be formed on the head, and the aperture can be formed in the proximal end of the stick or rods. The clip or axel can extend from the head and can be barrel-shaped or cylindrical, and bifurcated to form to halves or arms of the clip with a slot therebetween to allow the arms of the clip to resiliently and elastically bend or flex towards one another. The clip or arms thereof can have an outer diameter matching the inner diameter of the aperture. In addition, a flange, or flange halves, can be formed on distal ends of the clip or arms thereof. The flange can have an outer diameter greater than the inner diameter of the aperture to retain the clip or axel in the aperture. Furthermore, the arms, clip or axel can be longer than the aperture so that the flange is disposed outside of the aperture. The aperture can extend through the proximal end of the stick. The aperture can be formed an annular portion extending from the stick with the annular portion extending towards the concave portion and away from the longitudinal axis, and the aperture oriented transverse to the longitudinal axis and along the width of the stick. The ends of the clip or axel can be pressed together, aided by an inclined leading edge of the flanges of the arms, to insert the arms of the clip into and through the aperture. As the flanges clear the aperture, the resiliency and elasticity of the arms returns the arms to a parallel initial configuration with the flanges extending beyond the aperture both longitudinally and radially retain the clip in the aperture. A portion of the head opposite the flanges can also be enlarged with respect to the clip or axel to retain the aperture or annular portion of the stick between the flange and enlarged portion of the body. Thus, the enlarged portion and flange form a hub with an annular groove. In one aspect, the head can be removably coupled to the vent stick by pressing the arms of the clip together and withdrawing the clip from the aperture. One advantage of a removable head is that it allows a user to replace a vent stick or a polymer body after the scent has fully vented from the device, or to exchange it for a vent stick or a polymer body having a different desired scent, or the exchange different heads with respect to the stick or body.

In addition, the hinge can include one or more ridges corresponding to one or more depressions associated with the clip and the aperture to set or bias the orientation of the head with respect to the stick, and allowing the orientation to be changed. For example, the ridges can be formed in the clip or axel, while the depressions can be formed in the aperture. When the head and clip are rotated in the aperture, the clip compresses and the arms come together as a ridge is forced out of a depression. The one or more ridges and the one or more depressions define various different positions of the head with respect to the vent stick. When the head is pivoted to a different position, the ridge can enter a different depression and engage into the different position. The clip or axel rotating or pivoting in the aperture can define a pivot axis. The head and clip or axel can be formed of plastic.

It is contemplated that other materials or configurations may be also used to pivot the head with respect to the vent stick, such as joint configurations known in the art or bendable wire, rubber, or plastic materials and the like.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1. An air freshener device, comprising:
a) an elongated vent stick configured to extend into an air vent of a vehicle and having a proximal end, a distal end, and a longitudinal axis;
b) at least one scented body carried by the vent stick and configured to extend into the air vent;
c) a scent material interspersed throughout the at least one scented body and capable of dispersing out of the at least one body over time; and
d) a pair of transverse slots oriented transverse to one another and each bifurcating the vent stick in different direction to form four fingers oriented substantially parallel to one another.

2. A device in accordance with claim 1, wherein each of the pair of slots have a different length with respect to one another from the distal end of the vent stick.

3. A device in accordance with claim 1, wherein the pair of slots comprises:
a primary slot bifurcating only the vent stick to form a pair of vent rods forming a clip; and
a secondary slot bifurcating both the pair of vent rods of the vent stick and the at least one body.

4. A device in accordance with claim 3, wherein the at least one scented body further comprises:
a pair of scented bodies, each carried by a different one of the pair of vent rods, and with the primary slot separating the pair of bodies and the secondary slot bifurcating the pair of scented bodies.

5. A device in accordance with claim 1, further comprising:
a head formed on the proximal end of the vent stick and having a face configured to face outward from the air vent; and
the face having a concave cascading curvature from a top thereof to a bottom thereof.

6. A device in accordance with claim 5, further comprising:
the face having a lateral concave indentation oriented concave from side to side across the face.

7. A device in accordance with claim 5, wherein the cascading curvature is convex from the top of the face to the bottom of the face.

8. A device in accordance with claim 5, further comprising:
a channel formed in the bottom of the head behind the face and oriented transverse to the longitudinal axis of the vent stick.

9. A device in accordance with claim 5, wherein one of the pair of transverse slots is oriented horizontally and another of the pair of transverse slots is oriented vertically.

10. An air freshener device in combination with an air vent of a vehicle, the device comprising:
a) a vent stick extending into and engaging the air vent and having a proximal end, a distal end, and a longitudinal axis;
b) a pair of scented bodies carried by the vent stick and extending into the air vent;
c) a scent material interspersed within and throughout the pair of scented bodies and capable of dispersing out of the pair of scented bodies over time;
d) a primary slot oriented horizontally and extending into the vent stick from the distal end and bifurcating the vent stick to form a pair of vent rods forming a clip and separating the pair of scented bodies with each carried by a different one of the pair of vent rods; and
e) a secondary slot oriented vertically and extending into the vent stick from the distal end and oriented transverse to the primary slot and bifurcating the pair of vent rods and the pair of scented bodies to form four fingers oriented substantially parallel to one another.

11. A device in accordance with claim 10, wherein the primary and secondary slots have different lengths with respect to one another from the distal end of the vent stick.

12. A device in accordance with claim 10, further comprising:
a head formed on the proximal end of the vent stick and having a face configured to face outward from the air vent; and
the face having a concave cascading curvature from a top thereof to a bottom thereof.

13. A device in accordance with claim 12, further comprising:
the face having a lateral concave indentation oriented concave from side to side across the face.

14. A device in accordance with claim 12, wherein the cascading curvature is convex from the top of the face to the bottom of the face.

15. A device in accordance with claim 12, further comprising:
a channel formed in the bottom of the head behind the face and oriented transverse to the longitudinal axis of the vent stick.

16. An air freshener device, comprising:
a) an elongated vent stick configured to extend into an air vent of a vehicle and having a proximal end, a distal end, and a longitudinal axis;
b) a scent material associated with the vent;
c) a pair of transverse slots oriented transverse to one another and each bifurcating the vent stick in different direction to form four fingers oriented substantially parallel to one another; and
d) a head formed on the proximal end of the vent stick and having a face configured to face outward from the air vent.

17. A device in accordance with claim 16, wherein each of the pair of slots have a different length with respect to one another from the distal end of the vent stick.

18. A device in accordance with claim 16, further comprising:
at least one scented body carried by the vent stick and configured to extend into the air vent; and
the scent material being interspersed throughout the at least one scented body and capable of dispersing out of the at least one body over time.

19. A device in accordance with claim 18, wherein the pair of slots comprises:
a primary slot bifurcating only the vent stick to form a pair of vent rods forming a clip; and
a secondary slot bifurcating both the pair of vent rods of the vent stick and the at least one body.

20. A device in accordance with claim 16, further comprising:
a head formed on the proximal end of the vent stick and having a face configured to face outward from the air vent; and
the face having a concave cascading curvature from a top thereof to a bottom thereof.
